(19) United States
(12) Patent Application Publication
(10) Pub. No.: US 2016/0184027 A1
(43) Pub. Date: Jun. 30, 2016

(54) MAGNETIC EXCITATION SYSTEM AND METHOD FOR OPERATING THE SAME

(71) Applicant: METAL INDUSTRIES RESEARCH AND DEVELOPMENT CENTRE, Kaohsiung (TW)

(72) Inventors: Yan-Jun Chen, Kaohsiung (TW); Tung-Chieh Yang, Kaohsiung (TW); Yu-Fen Kuo, Kaohsiung (TW); Yu-Min Ting, Kaohsiung (TW); Ming-Hui Chen, Kaohsiung (TW); Tsung-Chih Yu, Kaohsiung (TW); Ho-Chung Fu, Kaohsiung (TW)

(21) Appl. No.: 14/626,979

(22) Filed: Feb. 20, 2015

Related U.S. Application Data
(60) Provisional application No. 62/098,095, filed on Dec. 30, 2014.

(51) Int. Cl. A61B 19/00; A61N 2/00

(52) U.S. Cl. CPC A61B 19/50; A61N 2/00; A61B 19/54; A61B 2019/504; A61B 2019/5404

(57) ABSTRACT

A magnetic excitation system includes a magnetic excitation apparatus for generating a magnetic field, and an analyzing device including a detecting unit for detecting magnetic flux of the magnetic field, and a processing unit. The processing unit is configured to: determine a magnetic flux distribution associated with a target according to the magnetic flux; generate, according to the magnetic flux distribution, a simulated magnetic field distribution over the target before a magnetic induction needle is punctured into the target; and calculate, in real time, temperature and ablating range associated with the target based on the magnetic flux when the magnetic induction needle is punctured into the target.

### FIELD OF THE INVENTION

The invention relates to a magnetic excitation system and a method for operating the magnetic excitation system.

### DESCRIPTION OF THE RELATED ART

Currently, magnetic thermal ablation has been widely utilized for treating tumor. Specifically, an alternating magnetic field is generated to pass through a target (e.g., parts of a human body that contain tumor tissues), while a magnetic induction needle is punctured into the target. The magnetic induction needle is affected by the alternating magnetic field and produces a resulting eddy current. In turn, the magnetic induction needle is heated by thermal energy produced by the eddy current, and is able to provide the heat necessary for thermal ablation or other operations such as cauterization.

However, during the process of thermal ablation, it is critical, yet difficult, to determine a temperature and an effective ablating range after the magnetic induction needle has been punctured into the target, especially when thermal ablation needs to be operated strictly within a certain range (e.g., the tumor tissue has a small size and is surrounded by normal tissues). At present, an operator has to determine when to stop the operation based on no more than his/her past experience.

### SUMMARY OF THE INVENTION

Therefore, the object of this invention is to provide a magnetic excitation system that is able to address the aforementioned drawbacks of the prior art.

Accordingly, a magnetic excitation system of this invention may include a magnetic excitation apparatus and an analyzing device.

The magnetic excitation apparatus is capable of generating a magnetic field.

The analyzing device includes at least one detecting unit configured to detect magnetic flux of the magnetic field passing therethrough, and a processing unit coupled communicatively to the at least one detecting unit.

The processing unit is configured to perform a simulation process for determining a magnetic flux distribution associated with a target, which is located within the magnetic field at a position corresponding to the at least one detecting unit, according to the magnetic flux detected by the at least one detecting unit before a magnetic induction needle is punctured into the target. The processing unit is configured to perform the simulation process further for generating, according to the magnetic flux distribution, a simulated magnetic field distribution associated with the target that would result from the magnetic induction needle being punctured into the target.

The processing unit is further configured to perform a real-time analysis process for calculating, in real time, a real-time magnetic field distribution associated with the target, and temperature and ablating range associated with the target based on the magnetic flux detected by the at least one detecting unit when the magnetic induction needle is punctured into the target.

Another object of this invention is to provide a method for operating the aforementioned magnetic excitation system.

Accordingly, a method of this invention may include the steps of:

positioning the detecting unit beside a target;

placing the electromagnetic excitation apparatus at a position corresponding to the target;

generating, by the electromagnetic excitation apparatus, a magnetic field that passes through the target and the detecting unit;

detecting, by the detecting unit, magnetic flux of the magnetic field passing therethrough; and

performing, by the processing unit, a simulation process for determining a magnetic flux distribution associated with the target according to the magnetic flux detected by the at least one detecting unit before a magnetic induct ion needle is punctured into the target, and for generating, according to the magnetic flux distribution, a simulated magnetic field distribution associated with the target that would result from the magnetic induction needle being punctured into the target.

### BRIEF DESCRIPTION OF THE DRAWINGS

Other features and advantages of the present invention will become apparent in the following detailed description of the embodiment with reference to the accompanying drawings, of which:

FIG. 1 is a schematic view of an embodiment of a magnetic excitation system being used on a target according to this invention;

FIG. 2 is a schematic view of a detecting unit of the magnetic excitation system according to this invention;

FIG. 3 is a flowchart of a method for operating the magnetic excitation system according to this invention; and

FIG. 4 is a schematic view of an alternative implementation of the embodiment, in which two detecting units are utilized, according to this invention.

### DETAILED DESCRIPTION OF THE EMBODIMENT

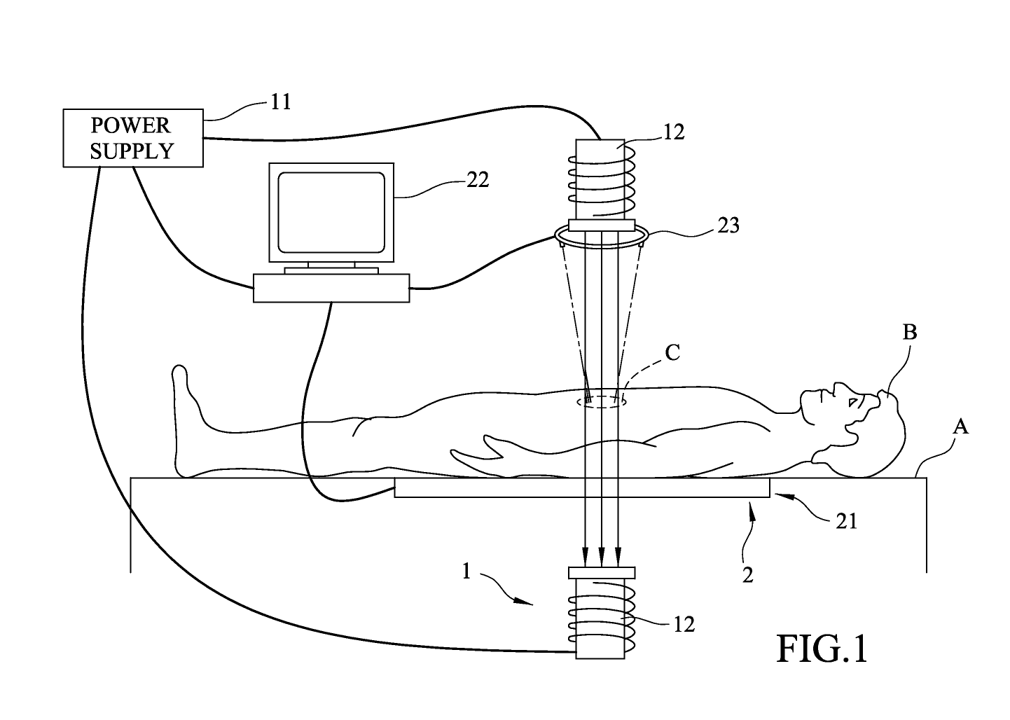

FIG. 1 illustrates an embodiment of a magnetic excitation system according to this invention. In this embodiment, the magnetic excitation system is for use on a patient (B) lying on a bed (A), and includes a magnetic excitation apparatus 1 and an analyzing device 2.

The magnetic excitation apparatus 1 includes a power supply 11, and a pair of induction coils 12 coupled to the power supply 11 for generating a magnetic field.

The analyzing device 2 includes at least one detecting unit 21, a processing unit 22 that is coupled communicatively to the detecting unit 21, and a marking device 23. The processing unit 22 is embodied as a computer in this embodiment for illustrative purposes, but should not be limited thereto in other embodiments of this invention.

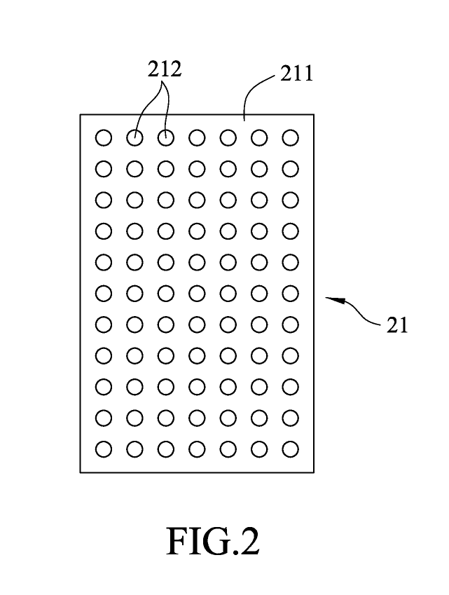

Further referring to FIG. 2, the detecting unit 21 includes a frame 211, and a plurality of fluxmeters 212 that are arranged on the frame 211 and spaced apart from each other. The detecting unit 21 may be embedded in the bed (A) and positioned below the patient (B).

Each of the fluxmeters 212 may be embodied using a magnetometer, an antenna or the like. The fluxmeters 212 are configured to detect magnetic flux of the magnetic field passing therethrough. The marking device 23 is coupled to and controlled by the processing unit 22, and includes alight source capable of illuminating a point.

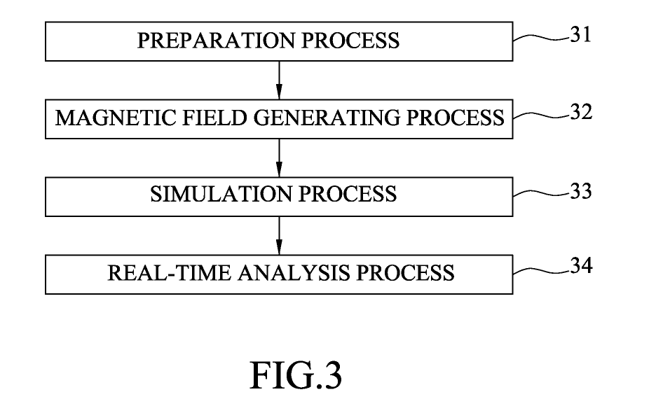

FIG. 3 illustrates processes of a method for operating the magnetic excitation system.

In a preparation process 31, the two induction coils 12 are placed coaxially on vertically opposite sides of the bed (A) at respective positions corresponding to a target (C) of the patient (B), such that the two induction coils 12 are also placed on vertically opposite sides of the lying patient (B) (i.e., the front and back sides). Specifically, the induction coils 12 are aligned with the target (C). The detecting unit 21 is mounted horizontally to the bed (A) and is positioned between the two induction coils 12, such that the frame 211 of the detecting unit 21 is beside the target (C). It is noted that, in other embodiments, the detecting unit 21 may be attached directly to the patient (B).

In a magnetic field generating process 32, the power supply 11 is turned on, enabling the two induction coils 12 to generate a magnetic field therebetween (i.e., passing through the detecting unit 21 and the target (C)). The fluxmeters 212 of the detecting unit 21 continuously detect the magnetic flux of the magnetic field passing through the detecting unit 21, and data regarding the magnetic flux is then transmitted to the processing unit 22.

In a simulation process 33 (i.e., before a magnetic induction needle (not shown) is actually punctured into the target (C)), the processing unit 22 determines a magnetic flux distribution associated with the target (C) according to the magnetic flux detected by the detecting unit 21 before the magnetic induction needle is punctured into the target (C). The operator is then allowed to select a location into which the magnetic induction needle is to be simulatively punctured.

In response to the selection of the location, the processing unit 22 generates a simulated magnetic field distribution associated with the target (C) that would result from a magnetic induction needle being punctured into the selected location of the target (C).

The operator may also input into the processing unit 22 other supportive data such as material of the magnetic induction needle, an intended depth to which the magnetic induction needle is to be punctured, information regarding the target (C), or a combination thereof, in order to assist the processing unit 22 to obtain better simulation results.

With the simulated magnetic field distribution available, the operator and/or the processing unit 22 may determine an optimal puncturing location that yields a desired result. After the optimal puncturing location is determined, the processing unit 22 is configured to control the marking unit 23 to mark the optimal puncturing location into which the magnetic induction needle should be punctured to reach the target (C) so as to assist the operator in accurately puncturing the magnetic induction needle.

In a real-time analysis process 34 (i.e., after the magnetic induction needle is actually punctured into the optimal puncturing location as marked by the marking unit 23), the detecting unit 21 continuously detects the magnetic flux and transmits the detected data to the processing unit 22. In the real-time analysis process 34, the processing unit 22 calculates, in real time, temperature and ablating range associated with the target (C) based on the magnetic flux detected by the detecting unit 21.

In particular, the calculation of the temperature associated with the target (C) as attributed to the magnetic field is described in the following.

By Faraday's law of induction, an electromotive force (EMF) attributed to change of the magnetic field can be calculated using

$$E = N \frac{d φ}{d i} ,$$

where E represents the EMF, N represents the turns of the induction coils 12, Φ represents the magnetic flux, and t represents time.

The magnetic flux Φ can be calculated using

Φ=∫Bds,

where B represents magnitude of the magnetic field (which can be detected by the detecting unit 21), and S represents an area of the surface on which the magnetic field passes.

In cases where the magnetic field cannot be directly detected (e.g., one that passes through the body of the patient (B)), with the current (I) available, the Biot-Savart Law can be used to approximate the magnetic field B(r):

$$B ( r ) = \frac{μ 0}{4 π} \int \frac{I d {I}^{′} ( r - {r}^{′} )}{{\vert r - {r}^{′} \vert}^{3}} ,$$

where μ₀ represents the magnetic constant, and (r-r′) represents the point where the magnetic field is computed.

Using the above data regarding the magnetic field, an approximated magnetic field on a particular height B(z) can be calculated using:

$$B ( z ) = \frac{{μ}_{0} I {a}^{2}}{2 {( {a}^{2} - {z}^{2} )}^{3 / 2}} \tilde{Z} .$$

With the magnetic field data now available, an eddy current (I) flowing through the magnetic induction needle can be calculated using Ampere's Law:

$$\int H d l = NI , = > I = \frac{1}{N} \int B d l ,$$

where H represents the magnetic field measured in units of amperes per meter (A/m), and dl represents an infinitesimal element.

Due to the skin effect, when current flows through the magnetic induction needle, a current density is largest near a surface and decreases within the magnetic induction needle. This in turn effectively increases an equivalent resistance of the magnetic induction needle and power dissipation (in the form of heat). As a result, the heat (Q) thus generated can be calculated using

Q=0.24I²Rt,

where I represents an equivalent current flowing through the magnetic induction needle, R represents an equivalent resistance of the magnetic induct ion needle, and t represents time.

In this embodiment, the heat dissipated due to heat transfer (thermal conduction, thermal radiation and convection) will be taken into consideration.

Using the Fourier's law, an outflow of heat from thermal conduction (Q_{cond}) can be calculated using:

$${Q}_{cond} = - kA \frac{d T}{d X} ,$$

where k represents thermal conductivity, A represents the heat transfer surface area, and dT/dX represents a temperature gradient.

Using the Newton's law of cooling, an outflow of heat from convection (Q_{conv}) can be calculated using:

Q_{conv.}=−hA(Tₛ−T_{∞}),

where h represents the heat transfer coefficient, A represents the heat transfer surface area, Tₛ represents the temperature on the surface of the magnetic induction needle, and T_{∞} represents the temperature of the environment (i.e., a place that is far away from the magnetic induction needle).

Using the Stefen-Bolzmann law, an outflow of heat from thermal radiation (Q_{rad.}) can be calculated using:

Q_{rad.}=−εσA(T_{S}⁴−T_{∞}⁴),

where ε represents the emissivity of the surface of the magnetic induction needle, and σ represents the Stefen-Bolzmann constant.

Moreover, internal heat (Q_{bio}) (i.e., heat generated from the biological activities within human body) may be calculated using:

Q_{bio}=ρ_{b}C_{b}ω_{b}(T_{b}−T)+Qₘₑₜ,

Where ρ_{b} represents the density of blood, C_{b} and ω_{b} are parameters regarding bloodflow, T_{b} represents a temperature on the target, and Qₘₑₜ represents heat generated through metabolism. The above parameters regarding the human body may be obtained from prior experimental results and/or from performing an (MRI) procedure on the human body.

Afterward, the heat equation can be used to calculate the heat distribution

$$ρ {C}_{P} \frac{\partial T}{\partial t} + ∇ \cdot ( - k ∇ T + ρ {C}_{P} Tu ) = Q$$

$$Q = 0.24 {I}^{2} Rt + {Q}_{bio} ,$$

where ρ represents mass density, Cₚ represents heat capacity, k represents heat conductivity, ∇T represents the temperature gradient, Tᵤ represents a heat transfer rate from convection.

On the other hand, the ablating range associated with the target (C) can be calculated using the following process.

In this embodiment, the target (C) is considered a concentric sphere with a body of normal tissues surrounding and enclosing the target (C) (i.e., the target (C) being a sphere with a radius R, the body being a sphere with an infinite radius, and tissues within the range of r where R≦r≦∞ are considered normal tissues).

It is assumed that a heat transfer equation regarding the body can be expressed as:

$$\frac{1}{{r}^{2}} {k}_{i} \frac{\partial }{\partial r} [ {r}^{2} ( \frac{\partial {T}_{i}}{\partial r} + {τ}_{T} \frac{{\partial }^{2} {T}_{i}}{\partial t \partial r} ) ] = ( 1 + {τ}_{ql} \frac{\partial }{\partial t} ) [ {ρ}_{i} {c}_{i} \frac{\partial {T}_{i}}{\partial t} - {q}_{rl} ] i = 1 , 2$$

where i=1 represents tissues of the target (C), i=2 represents normal tissues, ρᵢ represents a density of the tissues of type i, cᵢ represents a heat capacity of the tissues of type i, kᵢ represents a thermal conductivity of the tissues of type i, τ_{q} represents a relaxation time of thermal flux, and τ_{T} represents a relaxation time of temperature.

It is further assumed that at r=0, the temperature is a constant (dT₁/dr=0), at r=R, the temperature and a thermal flux of the normal tissues and the tissues of the target (C) are identical (i.e., T₁=T₂, q₁=q₂), and the temperature in the normal tissues (T₂, r=∞) is constant at 37° C. Using these assumptions as boundary conditions, the ablating range can be calculated.

One advantage of the real-time analysis process 34 is that since the temperature can be calculated, there is no need to attach an additional temperature sensor for measuring the temperature associated with the target (C) in real time.

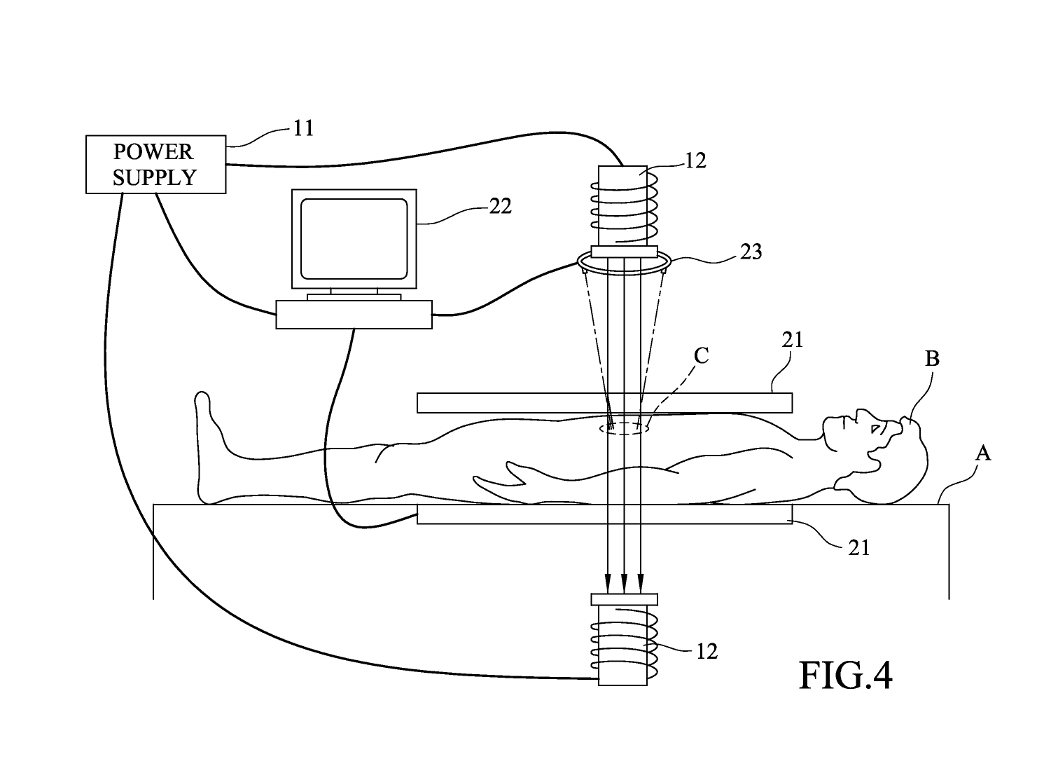

In an alternative implementation (see FIG. 4), the analyzing device 2 includes two detecting units 21. The two detecting units 21 are vertically spaced apart from each other so as to allow the target (C) to interpose therebetween.

In this implementation, the magnetic flux can be detected from two different heights. Therefore, in the simulation process 33, the processing unit 22 may determine a three-dimensional magnetic flux distribution associated with the target (C) according to the magnetic flux detected by the two detecting units 21, and may generate a three-dimensional simulated magnetic field distribution associated with the target (C) that would result from the magnetic induction needle being punctured into the target (C).

It is noted that for achieving a more accurate simulation, additional detecting units 21 may be placed between the induction coils 12 for obtaining more magnetic flux data for simulation. In another embodiment where only one detecting unit 21 is employed, the detecting unit 21 may be driven to move along a direction of the magnetic field (i.e., a direction perpendicular to a surface of the frame 211 that confronts the patient (B)) for obtaining more magnetic flux data for simulation. Moreover, an ultrasonic scanning apparatus (not depicted in the drawings) may be employed to obtain information regarding the target (C) and to provide the information thus obtained to the processing unit 22.

Further, in the real-time analysis process 34, the processing unit 22 may calculate a three-dimensional temperature distribution and a three-dimensional ablating based on the magnetic flux detected by the detecting units 21 when the magnetic induction needle is punctured into the target (C).

To sum up, the magnetic excitation of this invention employs the detecting unit(s) 21 for detecting the magnetic flux passing therethrough, and enables the processing unit 22 to generate the simulated magnetic field distribution associated with the target (C) that would result from a magnetic induction needle being punctured into the target (C) before the magnetic induction needle is actually punctured. The simulated temperature and ablating range associated with the target (C) may enable the operator and/or the processing unit 22 to determine an optimal puncturing location for puncturing of the magnetic induction needle, and the marking unit 23 is controlled to mark the optimal puncturing location to facilitate accurate puncturing of the magnetic induction needle at the optical puncturing location.

Furthermore, as the thermal ablation is in progress, the temperature and ablating range associated with the target (C) may be continuously monitored by the real-time analysis process 34. As a result, the operator has now an analytic basis, instead of past experience, as a guidance to determining when to stop performing the thermal ablation.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A magnetic excitation system comprising:
a magnetic excitation apparatus that is capable of generating a magnetic field; and
an analyzing device that includes at least one detecting unit configured to detect magnetic flux of the magnetic field passing therethrough, and a processing unit coupled communicatively to said at least one detecting unit;
wherein said processing unit is configured to perform a simulation process for
determining a magnetic flux distribution associated with a target, which is located within the magnetic field at a position corresponding to said at least one detecting unit, according to the magnetic flux detected by said at least one detecting unit before a magnetic induction needle is punctured into the target, and
generating, according to the magnetic flux distribution, a simulated magnetic field distribution associated with the target that would result from the magnetic induction needle being punctured into the target.

2. The magnetic excitation system of claim 1, wherein said processing unit is further configured to perform a real-time analysis process for calculating, in real time, a real-time magnetic field distribution associated with the target, and temperature and ablating range associated with the target based on the magnetic flux detected by said at least one detecting unit when the magnetic induction needle is punctured into the target.

3. The magnetic excitation system of claim 1, wherein said at least one detecting unit includes a frame that is to be positioned beside the target, and a plurality of fluxmeters that are arranged on said frame and that are spaced apart from each another.

4. The magnetic excitation system of claim 1, wherein said at least one detecting unit includes two detecting units, each of said detecting units including a frame, and a plurality of fluxmeters arranged on said frame and spaced apart from each another, said frames of said detecting units being spaced apart from each other to allow the target to interpose therebetween.

5. The magnetic excitation system of claim 1, wherein said analyzing device further includes a marking device that is coupled to and controlled by said processing unit and that is configured to mark a position, into which the magnetic induction needle should be punctured so as to reach the target, according to the simulated magnetic field distribution generated by said processing unit.

6. The magnetic excitation system of claim 5, wherein said magnetic excitation apparatus includes a power supply, and a pair of induction coils coupled to said power supply for generating the magnetic field.

7. A method for operating a magnetic excitation system, the magnetic excitation system including a magnetic excitation apparatus that is capable of generating a magnetic field, and an analyzing device that includes a detecting unit and a processing unit coupled communicatively to the detecting unit, said method comprising the steps of:
positioning the detecting unit beside a target;
placing the magnetic excitation apparatus at a position corresponding to the target;
generating, by the electromagnetic excitation apparatus, a magnetic field that passes through the target and the detecting unit;
detecting, by the detecting unit, magnetic flux of the magnetic field passing therethrough; and
performing, by the processing unit, a simulation process for
determining a magnetic flux distribution associated with the target according to the magnetic flux detected by the detecting unit before a magnetic induction needle is punctured into the target, and
generating, according to the magnetic flux distribution, a simulated magnetic field distribution associated with the target that would result from the magnetic induction needle being punctured into the target.

8. The method of claim 7, further comprising, after the step of performing the simulation process, the step of:
performing, by the processing unit, a real-time analysis process for calculating, in real time, a real-time magnetic field distribution associated with the target, and temperature and ablating range associated with the target based on the magnetic flux detected by the detecting unit when the magnetic induction needle is punctured into the target.

9. The method of claim 8, the analyzing device including two detecting units, wherein:
in the step of positioning the detecting unit, the detecting units are placed to be spaced apart from each other to allow the target to be interposed therebetween;
in the step of performing the simulation process, the processing unit is configured to determine a three-dimensional magnetic flux distribution associated with the target according to the magnetic flux detected by the two detecting units before the magnetic induction needle is punctured into the target, and to generate a three-dimensional simulated magnetic field distribution associated with the target that would result from the magnetic induction needle being punctured into the target; and
in the step of performing the real-time analysis process, the processing unit is configured to calculate three-dimensional temperature distribution and ablating range associated with the target based on the magnetic flux detected by the detecting units when the magnetic induction needle is punctured into the target.

10. The method of claim 7, wherein:
in the step of detecting the magnetic flux, the detecting unit is driven to move along a direction of the magnetic field;
in the step of performing the simulation process, the processing unit is configured to determine a three-dimensional magnetic flux distribution associated with the target according to the magnetic flux detected by the detecting unit before the magnetic induction needle is punctured into the target, and to generate a three-dimensional simulated magnetic field distribution associated with the target that would result from the magnetic induction needle being punctured into the target; and
in the step of performing the real-time analysis process, the processing unit is configured to calculate three-dimensional temperature distribution and ablating range associated with the target based on the magnetic flux detected by the detecting unit when the magnetic induction needle is punctured into the target.

11. The method of claim 7, the analyzing device further including a marking device that is coupled to and controlled by the processing unit, said method further comprising, after the step of performing the simulation process, the step of:
marking, by the marking device, a position, into which the magnetic induction needle should be punctured to reach the target, according to the simulated magnetic field distribution generated by the processing unit.

12. The method of claim 7, wherein, in the step of performing the simulation process, the processing unit generates the simulated magnetic field distribution based further on material of the magnetic induction needle, an intended depth to which the magnetic induction needle is to be punctured, and information regarding the target.
